(19) United States
(12) Patent Application Publication
(10) Pub. No.: US 2024/0050429 A1
(43) Pub. Date: Feb. 15, 2024

(54) PAZOPANIB PHARMACEUTICAL COMPOSITION, INJECTION AND PREPARATION METHOD AND USE THEREOF

(71) Applicant: Qx Therapeutics, Inc., Branford, CT (US)

(72) Inventors: Xiaofeng LI, Farmington, CT (US); Ho Yin Lo, Bethel, CT (US)

(21) Appl. No.: 17/885,503

(22) Filed: Aug. 10, 2022

(51) Int. Cl. A61K 31/506; A61K 47/40; A61K 47/10; A61K 47/26; A61K 9/00

(52) U.S. Cl. CPC A61K 31/506; A61K 47/40; A61K 47/10; A61K 47/26; A61K 9/0019

(57) ABSTRACT

Disclosed are a pazopanib pharmaceutical composition, an injection and a preparation method and the use thereof. The pharmaceutical composition comprises the following components: pazopanib, a cyclodextrin solubilizer and a solvent, wherein the mass ratio of the pazopanib to the cyclodextrin solubilizer is 1:(13 to 100), and based on 1 mL of the pharmaceutical composition, the concentration of the cyclodextrin solubilizer in the pharmaceutical composition is 6 to 330 mg/mL. The pharmaceutical composition prepared in the present disclosure can be used for treating diseases such as acute lung injury, pulmonary fibrosis and acute respiratory distress syndrome, and the therapeutic purpose can be achieved by using only a small amount of pazopanib; and the pharmaceutical composition has a high bioavailability, a high stability and a low impurity content, and there is no occurrence of drug accumulation phenomenon.

### CROSS-REFERENCE TO RELATED APPLICATIONS

The present application had a corresponding Chinese patent application 202110267979.9 filed on Mar. 11, 2021, which was published on Sep. 7, 2021, and the applicant was the same as the applicant of the present application. This Chinese patent application is incorporated herein by reference in its entirety.

### TECHNICAL FIELD

The present disclosure relates to a pazopanib pharmaceutical composition, an injection and a preparation method and the use thereof.

### BACKGROUND

Pazopanib hydrochloride is an oral VEGF-2 inhibitor developed by GlaxoSmithKline. Pazopanib hydrochloride also has inhibitory effects against both PDGFR and c-KIT tyrosine kinase, not only for renal cell carcinoma, but also for non-small cell carcinoma, breast cancer, sarcoma and other tumors.

Chinese patent document (CN 102970871 A) discloses a pharmaceutical composition and a preparation method therefor. The pharmaceutical composition comprises 10 mg/ml of pazopanib; 2% to 13% by weight of modified cyclodextrin, the modified cyclodextrin being selected such that the pKa of pazopanib with the modified cyclodextrin in water is lower than the pKa of pazopanib alone in water; a pH of 3.5 to 5.7; a tonicity adjusting agent as needed to provide an osmolality of 200 to 400 mOsm; and water. The pharmaceutical composition is only applied to eye drops, and there is no introduction of the treatment of acute lung injury. Moreover, the concentration of pazopanib hydrochloride in the pharmaceutical composition disclosed in the patent is too high, and after pazopanib hydrochloride being dissolved and clarified for a period of time, precipitates will be separated out, which will significantly reduce the efficacy of the drug.

Acute lung injury (ALI) can be induced by severe infections, hyperoxia, external injuries, drugs, mechanical ventilation, or stimulation of seawater and other factors, which is mainly manifested as infiltration of a large number of inflammatory cells such as neutrophils and macrophages, and barrier function disorders of pulmonary capillary endothelial and alveolar epithelial cells, and in severe cases, develops into acute respiratory distress syndromes (ARDS). It is reported that acute lung injury accounts for approximately 10% of intensive care unit admissions worldwide, with a mortality rate of up to 40%. Moreover, ischemia reperfusion (IR) is also one of the pathogenic factors of acute lung injury.

Chinese patent document (CN 109793740 A) discloses a use of pazopanib hydrochloride in the preparation of a drug for treating pulmonary fibrosis. The main causes of pulmonary fibrosis are the persistent damage and repeated repair of alveolar epithelial cells, the proliferation of myofibroblasts and fibroblasts and the deposition of a large amount of extracellular matrix secreted by the myofibroblasts and fibroblasts, which provides a microenvironment for pulmonary fibrosis. The repeated damage and repair of the above-mentioned lung tissues eventually lead to massive collagen deposition and pulmonary fibrosis in lung tissues.

In conclusion, although it is disclosed in the prior art that pazopanib can be used for treating pulmonary fibrosis, when pazopanib is used for treating pulmonary fibrosis, it can only be administrated orally, and there is a defect in the prior art that a larger amount of pazopanib is required for the treatment of pulmonary fibrosis. At present, there is an urgent need for a regimen that can achieve comparable efficacy with only a small amount of pazopanib.

### BRIEF SUMMARY OF THE INVENTION

The technical problem to be solved by the present disclosure is that when pazopanib is administrated orally to treat pulmonary fibrosis, there is a defect in the prior art that a larger dose is needed to achieve therapeutic purposes. The present disclosure provides a pharmaceutical composition, an injection, and a preparation method and the use thereof. The present disclosure provides a pazopanib pharmaceutical composition prepared by the present disclosure can be used for treating diseases such as acute lung injury, pulmonary fibrosis and acute respiratory distress syndrome, and the purposes of treating acute lung injury, pulmonary fibrosis and acute respiratory distress syndrome can be achieved by using only a small amount of the drug; and the pharmaceutical composition has a high bioavailability, a high stability and a low impurity content, and there is no occurrence of drug accumulation phenomenon.

The present disclosure provides a use of pazopanib in the preparation of a drug for treating acute lung injury.

In the present disclosure, pazopanib inhibits MAP3K2 and/or MAP3K3, thereby increasing active oxyradicals generated by neutrophils, and further achieving the purpose of treating acute lung injury. MAP3K2 refers to mitogen-activated protein kinase 2, and MAP3K3 refers to mitogen-activated protein kinase 3.

The present disclosure provides a pharmaceutical composition, comprising the following components: pazopanib, a cyclodextrin solubilizer and a solvent, wherein the mass ratio of the pazopanib to the cyclodextrin solubilizer is 1:(13 to 100), and based on 1 mL of the pharmaceutical composition, the concentration of the cyclodextrin solubilizer in the pharmaceutical composition is 6 to 330 mg/mL.

In the present disclosure, those skilled in the art know that the pazopanib is usually in the form of salts, generally pazopanib hydrochloride.

In the present disclosure, the cyclodextrin solubilizer is preferably a β-cyclodextrin derivative.

Wherein, the types of the β-cyclodextrin derivative preferably include one or more than one of hydroxypropyl-β-cyclodextrin, sulfobutyl ether-β-cyclodextrin and methyl-β-cyclodextrin, and more preferably include hydroxypropyl-β-cyclodextrin and/or sulfobutyl ether-β-cyclodextrin, such as hydroxypropyl-β-cyclodextrin.

In the present disclosure, those skilled in the art know that the solvent is usually water, such as water for injection. The water for injection is preferably free of oxygen. The water for injection is usually redistilled water.

In the present disclosure, the mass ratio of the pazopanib to the cyclodextrin solubilizer in the pharmaceutical composition is preferably 1:(15 to 40) or 1:(80 to 100), such as 1:15, 1:20, 1:33.26, 1:40, 1:50 or 1:100. More preferably, the mass ratio of the pazopanib to the cyclodextrin solubilizer is 1:(30 to 40), or 1:(90 to 100).

In the present disclosure, the concentration of the cyclodextrin solubilizer defined above complies with the national standard, i.e., of no more than 333 mg/mL.

In the present disclosure, the concentration of the cyclodextrin solubilizer is preferably 15 to 300 mg/mL, more preferably 30 to 40 mg/mL or 90 to 300 mg/mL, such as 90 to 200 mg/mL or 280 to 300 mg/mL, specifically 33.26 mg/mL, 100 mg/mL, 200 mg/mL or 300 mg/mL.

Wherein, when the cyclodextrin solubilizer includes hydroxypropyl-β-cyclodextrin, the concentration of the hydroxypropyl-β-cyclodextrin is preferably 15 to 300 mg/mL, more preferably 30 to 40 mg/mL or 90 to 300 mg/mL, such as 90 to 200 mg/mL or 280 to 300 mg/mL, specifically such as 33.26 mg/mL, 100 mg/mL, 200 mg/mL or 300 mg/mL.

In the present disclosure, the concentration of the pazopanib in the pharmaceutical composition is preferably 0.1 to 20 mg/mL, more preferably 0.5 to 2 mg/mL or 3 to 20 mg/mL, such as 1 mg/mL, 3 mg/mL, 4 mg/mL, 5 mg/mL or 20 mg/mL, more preferably 3 to 5 mg/mL.

When the pazopanib is pazopanib hydrochloride, the concentration of the pazopanib hydrochloride is preferably 0.1 to 20 mg/mL, more preferably 0.5 to 2 mg/mL or 3 to 20 mg/mL, such as 1 mg/mL, 3 mg/mL, 4 mg/mL, 5 mg/mL or 20 mg/mL, more preferably 3 to 5 mg/mL.

In the present disclosure, the pH value of the pharmaceutical composition is preferably 2.5 to 5.5, such as 3.25.

Wherein, in some preferred embodiments of the present disclosure, the pH value of the pharmaceutical composition itself has already been within the preferred range of 2.5 to 5.5, so the pharmaceutical composition does not contain a pH regulator.

Wherein, in other preferred embodiments of the present disclosure, the pH value of the pharmaceutical composition itself is not within the preferred range of 2.5 to 5.5, so an acid or alkali is further added as a pH regulator to obtain the preferred range of 2.5 to 5.5. Wherein, the types of the alkali in the pH regulator include one or more than one of aqueous ammonia, sodium hydroxide, sodium carbonate and sodium bicarbonate, and the types of the acid in the pH regulator include one or more than one of phosphoric acid, hydrochloric acid, citric acid and acetic acid.

In the present disclosure, the pharmaceutical composition can further comprise a conventional additive in the art, preferably comprises one or more than one of glycerol, propylene glycol, poloxamer, sucrose, mannitol, glucose, sodium chloride and amino acids, such as glucose and/or sodium chloride.

Wherein, based on 1 mL of the pharmaceutical composition, the concentration of the additive is preferably 0.1 to 200 mg/mL, more preferably 0.1 to 100 mg/mL, such as 0.9 or 5 mg/mL, much more preferably 0.9 to 5 mg/mL.

When the additive comprises glucose, based on 1 mL of the pharmaceutical composition, the concentration of the glucose is preferably 0.1 to 100 mg/mL, more preferably 4 to 6 mg/mL, such as 5 mg/mL.

When the additive comprises sodium chloride, based on 1 mL of the pharmaceutical composition, the concentration of the sodium chloride is preferably 0.1 to 100 mg/mL, more preferably 0.5 to 1.5 mg/mL, such as 0.9 mg/mL.

When the additive comprises mannitol, based on 1 mL of the pharmaceutical composition, the concentration of the mannitol is preferably 100 to 200 mg/mL.

In the present disclosure, the pharmaceutical composition preferably comprises the following components: pazopanib, a cyclodextrin solubilizer and a solvent, wherein the mass ratio of the pazopanib to the cyclodextrin solubilizer is 1:(15 to 100), and based on 1 mL of the pharmaceutical composition, the concentration of the cyclodextrin solubilizer in the pharmaceutical composition is 90 to 300 mg/mL; the types of the cyclodextrin solubilizer include hydroxypropyl-β-cyclodextrin and/or sulfobutyl ether-β-cyclodextrin.

In the present disclosure, the pharmaceutical composition preferably comprises the following components: pazopanib, a cyclodextrin solubilizer and a solvent, wherein the mass ratio of the pazopanib to the cyclodextrin solubilizer is 1:(15 to 40), and based on 1 mL of the pharmaceutical composition, the concentration of the cyclodextrin solubilizer in the pharmaceutical composition is 90 to 300 mg/mL; the types of the cyclodextrin solubilizer include hydroxypropyl-β-cyclodextrin and/or sulfobutyl ether-β-cyclodextrin.

In the present disclosure, the pharmaceutical composition preferably comprises the following components: pazopanib, a cyclodextrin solubilizer and a solvent, wherein the mass ratio of the pazopanib to the cyclodextrin solubilizer is 1:(90 to 100), and based on 1 mL of the pharmaceutical composition, the concentration of the cyclodextrin solubilizer in the pharmaceutical composition is 280 to 300 mg/mL; the types of the cyclodextrin solubilizer include hydroxypropyl-β-cyclodextrin and/or sulfobutyl ether-β-cyclodextrin.

In the present disclosure, the pharmaceutical composition preferably comprises the following components: pazopanib hydrochloride, hydroxypropyl-β-cyclodextrin and water, wherein the mass ratio of the pazopanib to the cyclodextrin solubilizer is 1:(15 to 100), and based on 1 mL of the pharmaceutical composition, the concentration of the hydroxypropyl-β-cyclodextrin in the pharmaceutical composition is 90 to 300 mg/mL.

In the present disclosure, the pharmaceutical composition preferably comprises the following components: pazopanib hydrochloride, hydroxypropyl-β-cyclodextrin and water, wherein the mass ratio of the pazopanib to the cyclodextrin solubilizer is 1:(15 to 40), and based on 1 mL of the pharmaceutical composition, the concentration of the hydroxypropyl-β-cyclodextrin in the pharmaceutical composition is 100 to 200 mg/mL.

In the present disclosure, the pharmaceutical composition preferably comprises the following components: pazopanib hydrochloride, hydroxypropyl-β-cyclodextrin and water, wherein the mass ratio of the pazopanib to the cyclodextrin solubilizer is 1:(90 to 100), and based on 1 mL of the pharmaceutical composition, the concentration of the hydroxypropyl-β-cyclodextrin in the pharmaceutical composition is 280 to 300 mg/mL.

In a preferred embodiment of the present disclosure, the pharmaceutical composition comprises the following components: pazopanib hydrochloride, hydroxypropyl-β-cyclodextrin and water for injection, and based on 1 mL of the pharmaceutical composition, pazopanib hydrochloride is 5 mg/mL, and hydroxypropyl-β-cyclodextrin is 200 mg/mL.

In a preferred embodiment of the present disclosure, the pharmaceutical composition comprises the following components: pazopanib hydrochloride, hydroxypropyl-β-cyclodextrin and water for injection, and based on 1 mL of the pharmaceutical composition, the concentration of pazopanib hydrochloride is 3 mg/mL, and the concentration of hydroxypropyl-β-cyclodextrin is 300 mg/mL.

In a preferred embodiment of the present disclosure, the pharmaceutical composition comprises the following components: pazopanib hydrochloride, hydroxypropyl-β-cyclodextrin, glucose, sodium chloride and water for injection, and based on 1 mL of the pharmaceutical composition, the concentration of pazopanib hydrochloride is 20 mg/mL, the concentration of hydroxypropyl-β-cyclodextrin is 300 mg/mL, the concentration of glucose is 5 mg/mL, and the concentration of sodium chloride is 0.9 mg/mL.

In a preferred embodiment of the present disclosure, the pharmaceutical composition comprises the following components: pazopanib hydrochloride, hydroxypropyl-β-cyclodextrin and water for injection, and based on 1 mL of the pharmaceutical composition, the concentration of pazopanib hydrochloride is 5 mg/mL, and the concentration of hydroxypropyl-β-cyclodextrin is 100 mg/mL.

In a preferred embodiment of the present disclosure, the pharmaceutical composition comprises the following components: pazopanib hydrochloride, hydroxypropyl-β-cyclodextrin and water for injection, and based on 1 mL of the pharmaceutical composition, the concentration of pazopanib hydrochloride is 4 mg/mL, and the concentration of hydroxypropyl-β-cyclodextrin is 200 mg/mL.

In a preferred embodiment of the present disclosure, the pharmaceutical composition comprises the following components: pazopanib hydrochloride, hydroxypropyl-β-cyclodextrin and water for injection, and based on 1 mL of the pharmaceutical composition, the concentration of pazopanib hydrochloride is 1 mg/mL, and the concentration of hydroxypropyl-β-cyclodextrin is 33.26 mg/mL.

The present disclosure also provides a method for preparing the above-mentioned pharmaceutical composition, the method comprising: mixing the above-mentioned pharmaceutical composition.

In the present disclosure, those skilled in the art know that the mixing generally meets the following requirement: the mixed solution of the pharmaceutical composition is clear. Being clear generally refers to the absence of particles visible to the naked eyes.

In the present disclosure, the mixing is performed in a conventional order in the art, preferably in which the pazopanib is added to the cyclodextrin solubilizer solution. The pazopanib is preferably added to the cyclodextrin solubilizer solution in a solid form. The concentration of the cyclodextrin solubilizer solution is preferably the concentration of the cyclodextrin solubilizer in the pharmaceutical composition.

In the present disclosure, the mixing is carried out by conventional means in the art, usually by stirring.

The stirring time is preferably 5 min or longer, such as 10 to 60 min, specifically 15 min, 20 min, 25 min.

The rotation speed of the stirring is preferably 250 rpm or higher, such as 250 to 500 rpm, such as 300 rpm, 350 rpm, 400 rpm.

After the stirring, the mixed solution of the pharmaceutical composition is preferably filtered with a microporous membrane. The pore size of the microporous membrane can be conventional in the art, preferably 0.22 to 0.45 m.

In the present disclosure, those skilled in the art know that, before the mixing, nitrogen and/or inert gas is usually introduced into the solvent so as to exclude oxygen from the solvent.

The time for introducing nitrogen can be conventional in the art, preferably 1 to 10 s.

In the present disclosure, those skilled in the art know that after the mixing, sterilization is further included. The sterilization is preferably a high-temperature sterilization. The high-temperature sterilization conditions can be conventional high-temperature sterilization conditions in the art.

Wherein, the sterilization is preferably carried out under nitrogen and/or inert gas.

Wherein, the temperature of the high-temperature sterilization is generally 115° C. or above, such as 115° C. or 121° C.

Wherein, the time for the high-temperature sterilization is preferably 8 min or longer, such as 8 to 60 min, specifically 8 min or 15 min.

The present disclosure also provides a use of the above-mentioned pharmaceutical composition in the preparation of a drug for treating one or more than one of acute lung injury, pulmonary fibrosis and acute respiratory distress syndrome.

The present disclosure also provides an injection, which contains the above-mentioned pharmaceutical composition.

The present disclosure also provides a method for preparing an injection, which is prepared by using the pharmaceutical composition according to the conventional method in the art. In the method for preparing the injection, the conventional method in the art generally comprises mixing various raw material components of the injection.

In the present disclosure, in the method for preparing the injection, preferably the pazopanib is added to the cyclodextrin solubilizer solution. Wherein, the pazopanib is preferably added to the cyclodextrin solubilizer solution in a solid form. The concentration of the cyclodextrin solubilizer solution is preferably the concentration of the cyclodextrin solubilizer in the pharmaceutical composition. Those skilled in the art know that after the pazopanib is added to the cyclodextrin solubilizer solution, a conventional additive and/or water for injection is usually added into the injection.

The present disclosure also provides a use of the above-mentioned injection in the preparation of a drug for treating one or more than one of acute lung injury, pulmonary fibrosis and acute respiratory distress syndrome.

On the basis of conforming to common knowledge in the art, the above-mentioned preferred conditions can be arbitrarily combined to obtain various preferred embodiments of the present disclosure.

Reagents and raw materials used in the present disclosure are all commercially available.

The positive effects of the present disclosure lie in: the pazopanib-containing pharmaceutical composition of the present disclosure has a good stability, a clear appearance and a low impurity content. When the pharmaceutical composition is used for treating acute lung injury, pulmonary fibrosis and acute respiratory distress syndrome, the therapeutic purposes can also be achieved by using only a relatively smaller amount of pharmaceutical ingredients; and the pharmaceutical composition has a low toxicity, there is no occurrence of drug accumulation phenomenon, and the pharmaceutical composition is well-tolerated at high concentrations.

### BRIEF DESCRIPTION OF THE DRAWINGS

FIG. 1 shows the effect of intravenous injection of the pharmaceutical composition at different doses 0.5 h before hydrochloric acid-induced acute lung injury on lung permeability.

### DETAILED DESCRIPTION OF THE INVENTION

The present disclosure is further described below by way of examples; however, the present disclosure is not limited to the scope of the described examples. For the experimental methods in which no specific conditions are specified in the following examples, selections are made according to conventional methods and conditions or according to the product instructions.

### Pazopanib Pharmaceutical Compositions

### Example 1 Pharmaceutical Composition and Preparation Method Therefor

1. Based on 1 mL of the pharmaceutical composition; the concentration of pazopanib hydrochloride in the pharmaceutical composition was 5 mg/mL, and the concentration of hydroxypropyl-β-cyclodextrin in the pharmaceutical composition was 200 mg/mL; the pH value of the pharmaceutical composition was 3.25.

2. The method for preparing the above-mentioned pharmaceutical composition specifically comprises the following steps:

(1) 5 mg of pazopanib hydrochloride was poured into the mixed solution in a container containing 1 mL of 200 mg/mL hydroxypropyl-β-cyclodextrin solution, wherein the solvent in the hydroxypropyl-β-cyclodextrin solution was water for injection. The concentration of pazopanib hydrochloride in the mixed solution was 5 mg/mL, and the concentration of hydroxypropyl-β-cyclodextrin in the mixed solution was 200 mg/mL, wherein nitrogen needed to be introduced into the water for injection for 30 min before use;

(2) The above-mentioned mixed solution was mixed until same was clear, the solution was filtered with a microporous membrane, nitrogen was introduced, a stopper was added, and a high-temperature sterilization was performed, wherein the rotation speed of the stirring was 250 rpm, the stirring time was 10 min, the pore size of the microporous membrane was 0.22 to 0.45 m, the time for introducing nitrogen was 1 to 10 s, and the high-temperature sterilization was performed under the condition of 121° C. for 15 min.

TABLE 1
Components and contents of the pharmaceutical compositions in
Examples 1 to 5 and Comparative Example 1

| | Concentration of | Types of | Concentration of | Mass ratio of pazopanib |
|---|---|---|---|---|
| | pazopanib | cyclodextrin | cyclodextrin solubilizer | hydrochloride to |
| | hydrochloride mg/mL | solubilizers | mg/mL | solubilizer |
| Example 1 | 5 | Hydroxypropyl-β- | 200 | 1:40 |
| | | cyclodextrin | | |
| Example 2 | 3 | Hydroxypropyl-β- | 300 | 1:100 |
| | | cyclodextrin | | |
| Example 3 | 20 | Hydroxypropyl-β- | 300 | 1:15 |
| | | cyclodextrin | | |
| Example 4 | 5 | Hydroxypropyl-β- | 100 | 1:20 |
| | | cyclodextrin | | |
| Example 5 | 1 | Hydroxypropyl-β- | 33.26 | 1:33.26 |
| | | cyclodextrin | | |

| | |
|---|---|
| Comparative | Oral administration of 33 mg of solid pazopanib hydrochloride from Formosa. |
| Example 1 | |

Note:
“/” indicated that the substance was not added. In each example, except the components of the pharmaceutical composition recorded in Table 1, the rest was water, making up to 1 mL.

The preparation methods and other parameters of the pharmaceutical compositions in the examples and comparative example in Table 1 were the same as those in Example 1. The pharmaceutical composition in each of the above-mentioned examples could be directly used as the dosage form of injection.

### Study of Components and Preparation Conditions

### Effect Example 1 Study on the Effect of Various Processes in the Preparation of Pazopanib Hydrochloride Injection on Stability

1. Types of Cyclodextrins

Two groups of pharmaceutical compositions with batch numbers of Q1 and Q2 were formulated respectively, wherein in both groups, the concentration of pazopanib hydrochloride was 4 mg/mL, and the concentration of cyclodextrin solubilizer was 200 mg/mL; and the cyclodextrin solubilizer in Q1 was hydroxypropyl-β-cyclodextrin, and in Q2 was sulfobutyl ether-β-cyclodextrin. Details were shown in Table 2 below, and other parameters in the two groups of pharmaceutical compositions and the parameters of the preparation methods were all the same as those in Example 1.

Content (%) of pazopanib hydrochloride was measured by high performance liquid chromatography: Column: ZORBAX Bonus-RP 4.6×150 mm, 3.5 μm; Column Temp.: room temperature; Flow Rate: 1.0 ml/min; Injection Volume: 10 μl; Detector: UV, λ=270 nm; Run Time: 15 min; Mobile Phase: 0.02 mol/L ammonium acetate solution (pH=6.8): ACN=65:35 (v/v); Needle wash/Diluent: 0.1% Perchloric acid:ACN=60:40 (v/v); isocratic elution.

Content (%) of impurities was measured by high performance liquid chromatography: Column: ZORBAX Bonus-RP 4.6×150 mm, 3.5 μm; Column Temp.: 40° C.; Flow Rate: 1.0 ml/min; Injection Volume: 10 μl; Detector: UV, λ=220 nm; Run Time: 56 min; Mobile Phase A: 0.1% perchloric acid; Mobile Phase B: acetonitrile; Needle wash/Diluent: 0.1% Perchloric acid:ACN=90:10 (v/v); Gradient elution:

| Time (min) | Mobile phase A (%) | Mobile phase B (%) |
|---|---|---|
| 0 | 98 | 2 |
| 8 | 77 | 23 |
| 13 | 77 | 23 |
| 45 | 20 | 80 |
| 50 | 20 | 80 |
| 51 | 98 | 2 |
| 56 | 98 | 2 |

TABLE 2

| | | Content (%) of pazopanib hydrochloride | | | | | | |
|---|---|---|---|---|---|---|---|---|
| | | | Day 14 | | | Day 28 | | |
| Batch | Solution | | | | 2° C. to | | | 2° C. to |
| number | appearance | Day 0 | 60° C. | 40° C. | 8° C. | 60° C. | 40° C. | 8° C. |
| Q1 | Clear | 99.2 | 103.0 | 104.1 | 101.4 | 101.8 | 102.8 | 102.6 |
| | under all | | | | | | | |
| | conditions | | | | | | | |
| Q2 | Clear | 99.4 | 102.7 | 97.1 | 99.4 | 101.0 | 99.8 | 100.0 |
| | under all | | | | | | | |
| | conditions | | | | | | | |

It could be seen from the above table that the content and solution appearance of the samples in Table 2 showed no significant changes after the samples were placed under 2° C. to 8° C., 40° C., and 60° C. for 1 month, respectively, and both of the samples could meet the clinical needs for administration. When the samples were placed at 60° C. for 1 month, the types of impurities increased, and the content of impurities tended to increase. The impurity RRT_{1.03} (1.03 referred to retention time) in the Q1 sample increased to 0.049%; the impurity in the Q2 sample increased to 0.176%.

2. Investigation on the Effect of the Concentration of Hydroxypropyl-β-Cyclodextrin on Solubility of Pazopanib Hydrochloride in the Pharmaceutical Composition

(1) Three pharmaceutical compositions were formulated and compared with Example 1, and the solution appearance and the content of the pazopanib hydrochloride on the day of formulation and 3 months later were examined when the pharmaceutical compositions were placed at 25° C. Details were shown in Table 3-1, the undisclosed parameters and preparation process of the three pharmaceutical compositions were the same as those in Example 1.

TABLE 3-1

| | | | | | Solution | |
|---|---|---|---|---|---|---|
| | | | | | appearance | |
| | | Cyclo- | | | when placed | Content % on |
| Batch | API | dextrin | Time for | | at 25° C. | the day of |
| number | (mg/mL) | (mg/mL) | dissolution | pH | for 3 months | formulation |
| Q3 | 5 | 20 | Undissolved | 3.22 | Turbid | 8.2 |
| | | | within 30 min | | | |
| Q4 | 5 | 50 | 7 min 30 s | 3.38 | Clear | 99.4 |
| Example 4 | 5 | 100 | 8 min 20 s | 3.24 | Clear | 100.6 |
| Example 1 | 5 | 200 | 8 min 15 s | 3.25 | Clear | 100.7 |

Note:
API referred to pazopanib hydrochloride.

(2) The solution appearance and content examined of pazopanib hydrochloride when the above-mentioned samples were placed for 28 days were shown in Table 3-2 below.

TABLE 3-2

| | 60° C. | | | 40° C. | | |
|---|---|---|---|---|---|---|
| Batch | Content | | Solution | Content | | Solution |
| number | (%) | pH | appearance | (%) | pH | appearance |
| Q4 | 79.6 | 2.671 | Solid | 93.7 | 3.011 | Solid |
| | | | precipitates | | | precipitates |
| | | | separated out | | | separated out |
| Example 4 | 103.1 | 3.317 | Clear | 102.2 | 3.272 | Clear |
| Example 1 | 101.3 | 3.328 | Clear | 101.6 | 3.303 | Clear |

(3) The detection and analysis of the impurity content in Examples 1 and 4 were performed, and the results were shown in Table 4 below. (0.96 in RRT_{0.96} referred to retention time).

TABLE 4

| | | | Impurity content (%) | | |
|---|---|---|---|---|---|
| | Cyclodextrin | Types of | 60° C. | | 40° C. |
| Batch number | (mg/ml) | impurities | Day 17 | Day 30 | Day 30 |
| Example 4 | 100 | RRT_{1.03} | 0.013 | 0.0626 | Not detected |
| | | RRT_{0.96} | 0.009 | 0.018 | 0.011 |
| Example 1 | 200 | RRT_{1.03} | 0.023 | 0.03 | 0.006 |
| | | RRT_{0.96} | 0.017 | 0.024 | 0.018 |

3. Investigation on pH

Pharmaceutical compositions with batch numbers of Q6 and Q7 were formulated respectively, and other parameters of the pharmaceutical compositions and the parameters of the preparation process were all the same as those in Example 1. Two pharmaceutical compositions with different pH were formulated for each batch number. In Q6 and Q7, the solubilizers were hydroxypropyl-β-cyclodextrin and sulfobutyl ether-β-cyclodextrin, respectively. The pH of the two pharmaceutical compositions was adjusted with hydrochloric acid solution/NaOH solution, and the stability at Day 0 and Day 14 was investigated at 60° C. Details were shown in Table 5:

TABLE 5

| | | | | | Content (%) of | Content (%) |
|---|---|---|---|---|---|---|
| | | | Solution appearance | | the pazopanib | of RRT_{1.03} |
| Batch | | Adjusted | Day 0, | 60° C., | hydrochloride | 60° C., |
| number | Cyclodextrin | pH | 60° C. | Day 14 | 60° C., Day 14 | Day 14 |
| Q6-1 | Hydroxypropyl- | Unadjusted pH | Clear | Clear | | 0.030% |
| Q6-2 | β-cyclodextrin | 3.0 | Clear | Clear | 101.8 | 0.038% |
| Q6-3 | | 9.1 | Clear | Solid | 11.6 | \ |
| | | | | precipitates | | |
| | | | | separated out | | |
| Q7-1 | Sulfobutyl | Unadjusted pH | Clear | Clear | | 0.103% |
| Q7-2 | ether-β- | 3.0 | Clear | Clear | 102.9 | 0.179% |
| Q7-3 | cyclodextrin | 5.5 | Clear | Clear | 102.0 | 0.028% |

Test results: It could be seen from Table 5 above that, the pharmaceutical composition without pH adjustment had lower content of single maximum unknown impurity (RRT_{1.03} impurity) than that with pH adjustment, and hydroxypropyl-β-cyclodextrin was selected through a solubilizer screening test.

In addition, the sample stability of samples with different pH after the high temperature sterilization was investigated.

Pharmaceutical compositions with batch numbers of Q8-1 and Q8-2 were formulated respectively, wherein, pH value of Q8-2 was adjusted to 4.0 with NaOH solution, pH value of Q8-1 was not adjusted, and other parameters of the pharmaceutical compositions and other parameters of the preparation process were the same as those in Example 4, and the appearance, content of the pazopanib hydrochloride of the samples before and after sterilization were investigated. Details were shown in Table 6 below:

TABLE 6

| | | | Content | | Batch | | |
|---|---|---|---|---|---|---|---|
| | | | before | | numbers | | Content after |
| Batch | | Solution | sterilization | Sterilization | after | Solution | sterilization |
| number | pH | appearance | (%) | conditions | sterilization | appearance | (%) |
| Q8-1 | 3.250 | Clear | 100.2 | 121° C., | Q02-36-3-3 | Clear | 100.5 |
| Q8-2 | 3.999 | Clear | 100.3 | 15 min | Q02-36-3-4 | Solid | 94.6 |
| | | | | | | precipitates | |
| | | | | | | separated out | |

In Table 6 above, the solution of which the pH was not adjusted was clear after sterilization, and no solid precipitates was separated out.

4. Investigation on Adding Order

Pharmaceutical compositions with batch numbers of Q9-1 and Q9-2 were formulated respectively, and other parameters of the pharmaceutical compositions and other parameters of the preparation process were the same as those in Example 4. The dissolution time, the solution appearance and content of the pazopanib hydrochloride were compared, and details were shown in Table 7 below.

TABLE 7

| Batch | | Dissolution | Solution | Content |
|---|---|---|---|---|
| number | Adding order | time | appearance | (%) |
| Q9-1 | API was poured into | 8 min 20 s | Clear | 100.6 |
| | hydroxypropyl-β- | | | |
| | cyclodextrin solution | | | |
| Q9-2 | hydroxypropyl-β- | 12 min | Clear | 102.4 |
| | cyclodextrin solution was | | | |
| | added into API | | | |

It could be seen from Table 7 above that, for the adding order, the dissolution time could be reduced by using the order in which the API was poured into the formulated hydroxypropyl-β-cyclodextrin solution.

5. Investigation on the Stirring Speed

Two groups of pharmaceutical compositions with different stirring speeds were formulated, and compared with Example 4. The parameters in the pharmaceutical compositions and the other parameters of the preparation process were the same as those in Example 4. As shown in Table 8 below, the dissolution time, appearance and content of the pazopanib hydrochloride at different stirring speeds of 250 rpm, 400 rpm and 150 rpm were investigated.

TABLE 8

| Batch | Stirring | Dissolution | | |
|---|---|---|---|---|
| number | speed | time | Solution appearance | Content (%) |
| Example 4 | 250 rpm | 8 min 20 s | Clear | 100.6 |
| Q10 | 400 rpm | 7 min | Clear | 102.1 |
| Q11 | 150 rpm | 1 h | Slightly turbid | 103.4 |

It could be seen from Table 8 above that, the lower the stirring speed, the longer the dissolution time for the sample, and the appearance of the sample at low speed was slightly turbid, so the rotational speed of the preparation process should not be lower than 250 rpm.

6. Investigation on Stirring Time

Three groups of pharmaceutical compositions with stirring time of 5 min, 10 min, and 20 min were formulated, respectively, and the other parameters were the same as those in Example 4. The stirring speed in the preparation process was 300 rpm, and the other parameters in the preparation process were the same as those in Example 4. As shown in Table 9 below, the solution appearance and content of the pazopanib hydrochloride were investigated.

TABLE 9

| Batch number | Stirring time | Solution appearance | Content (%) |
|---|---|---|---|
| Q12 | 5 min | Presence of undissolved | 101.0 |
| | | small solid particles | |
| Q13 | 10 min | Clear | 100.8 |
| Q14 | 20 min | Clear | 100.5 |

It could be seen from Table 9 above, undissolved small solid particles could also be observed in the sample at 5 min, and the sample was clear at 10 min, so the stirring time of the preparation process was not less than 10 min.

7. Investigation on Sterilization Process

Three groups of pharmaceutical compositions were formulated, and the other parameters were the same as those in Example 4. Three groups were subjected to 121° C. for 8 min, 121° C. for 15 min and 115° C. for 32 min, respectively. The other parameters in the preparation process were the same as those in Example 4. The appearance of the samples and changes in related substances before and after sterilization were investigated, and compared with Example 4. Data was shown in Table 10 below.

TABLE 10

| | | | | Content of the |
|---|---|---|---|---|
| | | | | pazopanib |
| | Sterilization | Solution | | hydrochloride |
| Batch number | conditions | appearance | pH | (%) |
| Q15 | Unsterilized | Clear | 3.245 | 100.6 |
| Q16 | 121° C., 8 min | Clear | 3.311 | 101.4 |
| Example 4 | 121° C., 15 min | Clear | 3.250 | 102.2 |
| Q17 | 115° C., 32 min | Clear | 3.293 | 100.5 |

It could be seen from Table 10 above, the samples were sterilized by different methods, the appearance and pH of the solution kept unchanged before and after the sterilization, the solution was clear, and no foreign substance was separated out; after sterilization, the number of detectable impurities increased. There was a great increase in the amounts of two impurities RRT_{0.9}6 and RRT_{1.08}. After sterilization at 121° C. for 8 min, the amounts of the two impurities were 0.012% and 0.015%. After sterilization at 121° C. for 15 min, the amounts of the two impurities were 0.010% and 0.010%, and there was no obvious difference, so the sterilization was carried out at 121° C. for 15 min.

For N₂ protection: the samples with N₂ protection and the samples without N₂ protection were subjected to a high-temperature sterilization, and then the changes in related substances in the samples were compared. The samples with N₂ protection showed low increase in impurities after sterilization. N₂ protection was required.

### Study of Toxicology

### Effect Example 2 Preparation of Samples for Preliminary Toxicology Experiment and Stability Study

1. Preparation of Samples for Preliminary Toxicology Experiment

In order to meet the requirements of the preliminary toxicological experiment, the concentration of pazopanib hydrochloride in the pharmaceutical composition was 20 mg/mL, the dosage of hydroxypropyl-β-cyclodextrin was 300 mg/mL, and other parameters and the parameters of the preparation process were the same as those in Example 1. The products were placed under the conditions of 60° C. and 2° C. to 8° C. for 30 days, respectively, and the solution appearance was a clear solution and there was no significant difference compared with Day 0. However, compared with Day 0, the number and total amount of impurities in the related substances placed at 60° C. for 30 days increased, and the impurity content of RRT_{1.03} increased to 0.028%, as shown in Table 11 below.

TABLE 11

| | Content of the pazopanib | | |
|---|---|---|---|
| | hydrochloride after | | Solution |
| Batch number | 30 days (%) | pH | appearance |
| Q18-60° C. | 91.5 | 2.957 | Clear |
| Q19-2-8° C. | 94.4 | 2.934 | Clear |

2. Pharmacokinetic Dosage Regimen

The specific dosage regimen was as shown in Table 12 and the specific pharmacokinetic test results were as shown in Table 13, and the mice were C57/BL male and female mice of 4 to 10 weeks old. Wherein, the blank control is saline, the pharmaceutical composition injected at different concentrations was the sample freshly prepared for preliminary toxicology experiment.

TABLE 12

| | | | Mode of treatment | | | | | | |
|---|---|---|---|---|---|---|---|---|---|
| | | | | | Dose of | | Adminis- | | |
| | Number | | | Order of | adminis- | Concen- | tration | Dose | |
| | of animals | | Test | adminis- | tration | tration | volume | rate (mL/ | Frequency/ |
| Groups | Male | Female | products | tration | (mg/kg) | (mg/mL) | (mL/kg) | kg/hr) | route/time |
| 1 | 2 | 2 | Blank | 1st | 0 | 0 | 5 | 20 | Single dose/ |
| | | | control | | | | | | intravenous/ |
| | | | | | | | | | 30 min |
| | | | | 2nd | 0 | 0 | 5 | 20 | Single dose/ |
| | | | | | | | | | intravenous/ |
| | | | | | | | | | 30 min |
| | | | | 3rd | 0 | 0 | 5 | 20 | Single dose/ |
| | | | | | | | | | intravenous/ |
| | | | | | | | | | 30 min |
| | | | | 4th | 0 | 0 | 5 | 20 | Single dose/ |
| | | | | | | | | | intravenous/ |
| | | | | | | | | | 30 min, for |
| | | | | | | | | | 5 days |
| 2 | 2 | 2 | Injections of | 1st | 10 | 2 | 5 | 20 | Single dose/ |
| | | | pharmaceutical | | | | | | intravenous/ |
| | | | compositions | | | | | | 30 min |
| | | | at different | 2nd | 30 | 6 | 5 | 20 | Single dose/ |
| | | | concentrations | | | | | | intravenous/ |
| | | | | | | | | | 30 min |
| | | | | 3rd | 90 | 18 | 5 | 20 | Single dose/ |
| | | | | | | | | | intravenous/ |
| | | | | | | | | | 30 min |
| | | | | 4th | 90 | 18 | 5 | 20 | Single dose/ |
| | | | | | | | | | intravenous/ |
| | | | | | | | | | 30 min, for |
| | | | | | | | | | 5 days. |

Phase 1: The 1^{st}, 2^{nd} and 3^{rd} administrations were carried out on the 1^{st}, 5^{th}, and 9^{th} days respectively, the administration time was 30 min, and the administration was performed by intravenous infusion;

Phase 2: The 4^{th} administration was carried out on the 29^{th} to 33^{rd} days, dosing every day for a total of 5 days, the administration time was 30 min, the administration was performed by intravenous infusion.

TABLE 13

| | | | | |
|---|---|---|---|---|
| T_{1/2} | Tₘₐₓ | Cₘₐₓ | AUC₍₀₋₂₄₎ | AUC_{(0-∞)} |
| hr | hr | ng/mL | hr*ng/mL | hr*ng/mL |

Group 2 Day 1 (10 mg/kg, continuous intravenous infusion)

| | | | | | |
|---|---|---|---|---|---|
| 201 (male) | 4 | 0.25 | 52844 | 83122 | 83893 |
| 202 (male) | 3 | 0.25 | 48227 | 130584 | 131705 |
| Average value | 4 | 0.25 | 50536 | 106853 | 107799 |
| 203 (female) | 3 | 0.25 | 52616 | 115759 | 116253 |
| 204 (female) | 4 | 0.25 | 57230 | 160437 | 162439 |
| Average value | 4 | 0.25 | 54923 | 138098 | 139346 |

Group 2 Day 5 (30 mg/kg, continuous intravenous infusion)

| | | | | | |
|---|---|---|---|---|---|
| 201 (male) | 4 | 0.25 | 137296 | 353609 | 356249 |
| 202 (male) | 4 | 0.25 | 129882 | 339847 | 342958 |
| Average value | 4 | 0.25 | 133589 | 346728 | 349604 |
| 203 (female) | 3 | 0.50 | 125423 | 258220 | 259050 |
| 204 (female) | 5 | 0.25 | 151128 | 454428 | 463132 |
| Average value | 4 | 0.38 | 138276 | 356324 | 361091 |

Group 2 Day 9 (90 mg/kg, continuous intravenous infusion)

| | | | | | |
|---|---|---|---|---|---|
| 201 (male) | 4 | 0.50 | 248303 | 1129547 | 1140827 |
| 202 (male) | 4 | 0.25 | 239703 | 1158150 | 1174083 |
| Average value | 4 | 0.38 | 244003 | 1143849 | 1157455 |
| 203 (female) | 4 | 0.25 | 231390 | 1114339 | 1128935 |
| 204 (female) | 4 | 0.25 | 269473 | 1157141 | 1174720 |
| Average value | 4 | 0.25 | 250432 | 1135740 | 1151827 |

It could be seen from the above data that, the pharmacokinetic parameters were positively correlated to dose, i.e., the larger the dose, the larger the area under the curve (AUC value) and the maximum plasma concentration (Cₘax value).

In addition, for each dose, there was little difference in Tₘₐₓ and T_{1/2}, and peaks appeared at about 0.25 h, and half-lives were at about 4 h. The AUC(0-24) values at the doses of 10 mg/kg, 30 mg/kg and 90 mg/kg presented a multiple relationship, which increased by a factor of about 3 times. It could be seen therefrom that there was no occurrence of drug accumulation phenomenon for the pharmaceutical composition injections of the present disclosure at different concentrations.

### Effect of the Pharmaceutical Composition

### Effect Example 3 Treatment of Acid-Induced Acute Lung Injury with the Pharmaceutical Composition

1. In this example, the doses of pazopanib hydrochloride were 1 mg/kgbw, 3 mg/kgbw, and 10 mg/kgbw, respectively, which corresponded to the freshly prepared pharmaceutical compositions in Example 2 (being freshly formulated referred to within 1 day after the formulation was completed), and the therapeutic effect of these doses on hydrochloric acid-induced acute lung injury in mice were determined, wherein the body weight of each mouse was 20 g.

8 to 10 weeks old C57/BL male and female mice were infused with 0.05 M HCl at 2.5 μl/bwg through the trachea. 4 hours later, 100 μl of FITC-labeled albumin (10 mg/ml) was injected through the posterior segment of eyeball. Finally, the mice were euthanized and lung tissue samples were collected 6 hours after the induction of injury to measure changes in pulmonary permeability and lung histopathology. The above-mentioned three doses of the pharmaceutical composition of Example 2 were administered via tail intravenous injection at 0.5 h and 2 h before injury and 0.5 h and 2 h after injury to observe the prophylactic and therapeutic effects. Meanwhile, plasma was collected and bronchoalveolar lavage was performed after mice were euthanized.

In the experiment for survival analysis, 0.1M HCl was injected at 2.5 μL/bwg into the mice through the trachea, and the characteristics of the mice were observed within 30 h. Survival analysis was performed using a kaplan-meier method, and statistical analysis was performed using log-rank test (or log-rank Mantel-Cox test), and the obtained P value was less than 0.0001, indicated statistical significance.

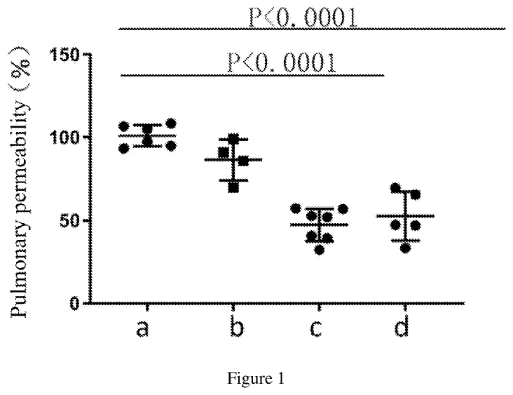

As shown in FIG. 1, the abscissa was the dose of intravenous injection at 0.5 hours before ALI was induced with hydrochloric acid, wherein a referred to the injection of hydroxypropyl-β-cyclodextrin, b, c, and d referred to the injection of 1 mg/kgbw, 3 mg/kgbw, 10 mg/kgbw of pazopanib hydrochloride, respectively, that was, corresponding to different volumes of the pharmaceutical composition of Example 2; the ordinate was the pulmonary permeability, and the specific test results were shown in Table 15. It could be obviously seen from FIG. 1 that, when 1 mg/kgbw of the pharmaceutical composition containing pazopanib hydrochloride was injected, the pulmonary permeability was significantly reduced, and when the injection amount was 10 mg/kgbw, the pulmonary permeability was still significantly reduced, which indicated that the pharmaceutical composition containing pazopanib hydrochloride was effective in preventing acute lung injury. The specific test results were as shown in Table 14 below.

TABLE 14

| | Percentage (%) of reduced |
|---|---|
| | lung permeability |
| 1 mg/kgbw | 2 to 30 |
| 3 mg/kgbw | 45 to 70 |
| 10 mg/kgbw | 35 to 65 |
| Comparative Example 1 | 45 to 70 |
| (1650 mg/kgbw) | |

2. In this example, the doses of pazopanib hydrochloride were 3 mg/kgbw and 10 mg/kgbw (mg/kgbw referred to the injection of 3 mg and 10 mg per 1 kg body weight of mice), respectively. The freshly formulated pharmaceutical composition in Example 5 was used. The therapeutic and preventive effects of these doses on hydrochloric acid-induced acute lung injury in mice were determined, wherein the body weight of each mouse was 20 g, and the mice were 8 to 10 weeks old C57/BL female mice.

The specific detection steps were as follows:
(1) the mice were anesthetized by subcutaneous injection of 100 mg/kg of ketamine and 10 mg/kg of xylazine, wherein mg/kg referred to the concentration in water for injection, and the water for injection was double distilled water;
(2) 30 min before inducing acute lung injury, the pharmaceutical composition in Example 5 was injected into the retroorbital sinus (RO) at doses of 3 mg/kgbw and 10 mg/kgbw, respectively; meanwhile, a blank control group was set up, and 33.26 mg/mL of hydroxypropyl-β-cyclodextrin was injected;
(3) the mice were fixed vertically from the incisors, the 22 G catheter was guided to 1.5 cm below the vocal cords, and 2.5 μL/g of 0.05 M HCl (50 μL) was instilled through the orotracheal tube to induce acute lung injury; the breathing of the mice was checked and the mice were placed on a heating pad;
(4) after 4 hours, 100 μl of FITC-albumin (10 mg/ml) was injected from RO;
(5) 6 hours after the induction of acute lung injury, the mice were euthanized to collect lung tissue samples—bronchoalveolar lavage fluid, and the proportion of FITC-labeled tissue components in the bronchoalveolar lavage fluid was tested, the test data for the doses of 3 mg/kgbw and 10 mg/kgbw, and the blank control group were shown in Table 15 (at a dose of 3 mg/kgbw) and Table 16 (at a dose of 10 mg/kgbw), respectively.

Meanwhile, a fluorescence spectrophotometer was used to detect the fluorescence intensity of the lung tissue samples in the experimental groups with the test excitation light wavelength of 485 nm and the emission light wavelength of 535 nm.

TABLE 15

| | | | | | | Average value of |
|---|---|---|---|---|---|---|
| | | Sample | Test | Repeated | Repeated | the test for each |
| | Test date | number | No. 1 | test No. 2 | test No. 3 | sample *100 (%) |
| Experimental | 2020 Jul. 3 | 1 | 0.27 | 0.28 | 0.26 | 26.94 |
| group | | 2 | 0.3 | 0.3 | 0.3 | 30.01 |
| | | 3 | 1.03 | 1.03 | 1.01 | 102.62 |
| | | 4 | 0.4 | 0.39 | 0.4 | 39.68 |
| | 2020 Jul. 1 | 5 | 0.45 | 0.42 | / | 43.31 |
| | | 6 | 1.26 | 1.3 | / | 128.03 |
| | | 7 | 0.42 | 0.41 | / | 41.41 |
| | | 8 | 1.16 | 1.03 | / | 109.35 |
| | | 9 | 0.53 | 0.5 | / | 51.54 |
| Average value of all tests in the experimental group | | | | | | 63.65 |
| Blank | 2020 Jul. 3 | 1 | 0.8 | 0.83 | 0.81 | 81.54 |
| control | | 2 | 0.96 | 0.96 | 0.99 | 97.09 |
| group | | 3 | 1.19 | 1.22 | 1.23 | 121.37 |
| | 2020 Jul. 1 | 4 | 1.26 | 1.19 | / | 122.9 |
| | | 5 | 1 | 1.03 | / | 101.66 |
| | | 6 | 0.87 | 0.85 | / | 86.24 |
| | | 7 | 1.15 | 1.17 | / | 116.28 |
| | | 8 | 0.72 | 0.73 | / | 72.92 |

| | |
|---|---|
| Average value of all tests in the blank control group | 100. |

Note:
p = 0.02891767, indicated that the experimental data was statistically significant; The experimental group referred to the pharmaceutical composition of Example 5 at a dose of 3 mg/kgbw; The average value for the experimental group and the blank control group (repeated 9 times and 8 times respectively, each test being repeated 2 to 3 times) was the lung permeability.

TABLE 16

| | | | | | | Average value of |
|---|---|---|---|---|---|---|
| | | Sample | Test | Repeated | Repeated | the test for each |
| | Test date | number | No. 1 | test No. 2 | test No. 3 | sample *100 (%) |
| Experimental | 2020 Aug. 24 | 1 | 0.55 | 0.56 | 0.58 | 56.55 |
| group | | 2 | 0.56 | 0.58 | 0.58 | 57.13 |
| | | 3 | 0.83 | 0.83 | 0.85 | 83.57 |
| | | 4 | 0.73 | 0.75 | 0.73 | 73.63 |
| | 2020 Sep. 4 | 5 | 0.29 | 0.29 | 0.3 | 29.38 |
| | | 6 | 0.46 | 0.45 | 0.49 | 46.67 |
| | | 7 | 0.47 | 0.41 | 0.5 | 45.96 |
| | | 8 | 0.93 | 0.92 | 0.93 | 92.47 |
| Average value of all tests in the experimental group | | | | | | 60.67 |
| Blank | 2020 Aug. 24 | 1 | 1.09 | 0.95 | 1.04 | 102.47 |
| control | | 2 | 0.73 | 0.72 | / | 72.28 |
| group | | 3 | 1.29 | 1.28 | / | 128.51 |
| | | 4 | 0.96 | 0.9 | 1.03 | 96.74 |
| | 2020 Sep. 4 | 5 | 0.8 | 0.8 | 0.74 | 77.94 |
| | | 6 | 0.66 | 0.65 | 0.65 | 65 |
| | | 7 | 0.73 | 0.75 | 0.73 | 73.71 |
| | | 8 | 1.02 | 0.93 | 1.02 | 98.72 |
| | | 9 | 1.85 | 1.84 | 1.85 | 184.62 |

| | |
|---|---|
| Average value of all tests in the blank control group | 100 |

Note:
p = 0.01921189, indicated that the experimental data was statistically significant; the experimental group referred to the pharmaceutical composition of Example 5 at a dose of 10 mg/kgbw.

It could be seen from Table 15 and Table 16 above that, before the acute lung injury in the mice was induced with hydrochloric acid, the pharmaceutical composition in Example 5 of the present disclosure was injected into the mice. Compared with the blank control group, the pulmonary permeability of the mice in the experimental group was significantly lower than 100% of the blank control group. The pulmonary permeability of the mouse was 63.65% when the dose was 3 mg/kgbw, and the pulmonary permeability of the mouse was 60.67% when the dose was 10 mg/kgbw. It could be concluded that the pharmaceutical composition in Example 5 of the present disclosure could effectively prevent and treat acute lung injury.

### Study of Pharmacokinetics

### Effect Example 4

Comparison of the characteristics of rats and mice injected with the pharmaceutical compositions in Example 1.

The experimental parameters were as follows:
Mice: 8 to 10 weeks old female mice
Components of the pharmaceutical composition: Pazopanib hydrochloride:hydroxypropyl-β-cyclodextrin:water (0.8%:33.26%:65.94%)
Concentration of stock solution: 8 mg/mL
Working concentration: 0.4 mg/mL
Route of administration: intravenous injection
Injection volume: intravenous injection 100 μL
Time point of blood collection: 5 min, 15 min, 30 min, 1 h, 2 h, 4 h, 8 h, 12 h, 24 h
Plasma collection: 200 μL of plasma was collected from the posterior segment of eyeball into a BD lithium heparin w/plasma separation tube, and the collected plasma was rotated and then stored at −80° C. Wherein, the plasma was collected for 5 min below zero and then rotated (90 s, 12000 g). The test results were shown in Table 17 below.

TABLE 17
Parmacodynamic data after injection of the pharmaceutical compositions
in Example 1 in rats and mice

| | Rats | Mice |
|---|---|---|
| Dose of administration (mg/kg) | 3 | 2 |
| AUC-for 24 h (mg*h/L) | 44.7 | 137 |
| AUC-infinity (mg*h/L) | 45.3 | 149 |
| Mean residence time (h) | 4.3 | 8.5 |
| Half-life period (h) | 3.0 | 5.9 |
| Clearance (L*h/kg) | 0.066 | 0.013 |
| Distribution volume (L/kg) | 0.29 | 0.11 |
| Co (Cmax) (mg/L) | 17.8 | 27.8 |

NOTE:
The doses of administration were the concentration of the pharmaceutical composition corresponding to the injection of 3 mg of pazopanib hydrochloride in a rat with a body weight of 1 kg, and the concentration of the pharmaceutical composition corresponding to the injection of 2 mg of pazopanib hydrochloride in a mouse with a body weight of 1 kg.

It could be seen from Table 17, the distribution volume in both rats and mice was less than 1 L/kg, indicated that the pharmaceutical composition in Example 1 had a low tissue permeability; rats and mice also had lower clearance.

The data of plasma concentration at each time point for blood collection in rats were shown in Table 18 below:

TABLE 18

| Time point | Plasma | Analyte | IS peak | | Calculated |
|---|---|---|---|---|---|
| of blood | concentration | peak area | area | | concentration |
| collection | (g/L) | (× 10⁵) | (× 10⁴) | Area ratio | (μg/mL) |
| 5 min | 15.0 | 2.64 | 1.26 | 20.9524 | 15.00 |
| 15 min | 10.6 | 1.79 | 1.20 | 14.9167 | 10.60 |
| 30 min | 9.8 | 1.73 | 1.25 | 13.8400 | 9.82 |
| 1 h | 6.3 | 1.17 | 2.10 | 5.5714 | 6.33 |
| 3 h | 4.5 | 9.90 | 1.51 | 6.5563 | 4.49 |
| 7 h | 1.5 | 4.53 | 1.90 | 2.3842 | 1.46 |
| 24 h | 0.1 | 5.59 | 1.52 | 0.3678 | 0.00 |

The data of drug concentration in plasma at each time point for blood collection in mice were shown in Table 19 below:

TABLE 19

| Time point of | drug concentration |
|---|---|
| blood collection | in plasma (g/L) |
| 5 min | 26.7 |
| 15 min | 18.4 |
| 30 min | 23.7 |
| 1 h | 19.4 |
| 2 h | 12.6 |
| 4 h | 10.3 |
| 9 h | 5.4 |
| 15 h | 2.3 |
| 24 h | 1.2 |

### Stability Assay of Clinical Batches

### Effect Example 5

Stability testing of clinical batch of product code 033-160A Pazopanib Hydrochloride Injection (5 mg/mL), lots included in scope: K-20-097, parameters of the pharmaceutical compositions and preparation process were the same as those in Example 1.

All stability was tested by the stability program, which indicated critical quality attributes to assess product degradation. Samples was stored in both upright (back up) and inverted orientations at the following conditions shown in Table 20 and tested at the following time points, data were shown in Tables 21-23.

Due to that the operations were done in different instruments and based on different operating parameters, there is a little difference in the RRT value (about ±0.1), but it is understood that the difference is common and reasonable in the art, it will not influence the person skilled in the art to discriminate whether two RRT values refer to the same materials.

TABLE 20
Storage condition and Testing Intervals

| Storage Condition | Testing Intervals |
|---|---|
| 5° C. ± 3° C., Inverted orientation | 0, 3, 6, 9 months |
| 5° C. ± 3° C., Upright orientation | 0, 9 months |
| 25° C. ± 2° C./60% RH ± 5%, Inverted orientation | 0, 1, 2, 3, 6 months |

The abbreviations in the Effect Example 5 are as follows:
NA=Not Applicable
ND=Not Detected
NMT=Not more than
LOQ=Limit of Quantitation
B, M, E=Beginning, Middle, End
DCP=2,4-dichloropyrimidine
AMBF=5-amino-2-methylbenzene-sulfonamide
Pazo-1=N-(2-Chloropyrimidin-4-yl)-2,3-dimethyl-2H-indazol-6-amine
Pazopanib-C2=5-((2-((2,3-dimethyl-2H-indazol-6-yl)methylamino)pyrimidin-4-yl-amino)-2-methylbenzenesulfonamide: hydrochloride
DMIA·HCl=2,3-dimethyl-2H-indazol-6-amine hydrochloride
Pazo-2=N-(2-chloropyrimidin-4-yl)-N,2,3-trimethyl-2H-indazol-6-amine
DMIA·HCl-P=2,3-dimethyl-6-nitro-2H-indazole
AMBF-P=2-methyl-5-nitrobenzene-sulfonamide.

Methods in Tables 21-23 are as follows:

Method GRAM-TM-0003: (1) view sample through a clear container; (2) examine the sample for any particulate or foreign matter; (3) place sample in front of white background and view for characteristics only distinguishable against a white background; (4) place sample in front of black background and view for characteristics only distinguishable against a black background.

Method GRAM-TM-0017: (1) following calibration the probe of pH Meter will enter measurement mode; (2) place the probe into the sample solution so it is immersed; (3) allow the pH to settle; (4) record the pH and temperature in associated lab note notebook; (5) when all sampling is complete, rinse the electrode, blot dry, and store in appropriate solution.

Method GRAM-ATM-1060: Column: Agilent ZORBAX Bonus-RP 4.6×150 mm, 3.5 μm; Column Temp.: room temperature; Flow Rate: 1.0 ml/min; Injection Volume: 10 μl; Detector: UV, λ=270 nm; Run Time: 15 min; Mobile Phase: 0.02 mol/L ammonium acetate solution (pH=6.8): ACN=65:35 (v/v); Needle wash/Diluent: 0.1% Perchloric acid:ACN=60:40 (v/v); isocratic elution.

Method GRAM-ATM-1063: Column: ZORBAX Bonus-RP 4.6×150 mm, 3.5 μm; Column Temp.: 40° C.; Flow Rate: 1.0 ml/min; Injection Volume: 10 μl; Detector: UV, λ=220 nm; Run Time: 56 min; Mobile Phase A: 0.1% perchloric acid; Mobile Phase B: acetonitrile; Needle wash/Diluent: 0.1% Perchloric acid:ACN=90:10 (v/v); Gradient elution:

| Time (min) | Mobile phase A (%) | Mobile phase B (%) |
|---|---|---|
| 0 | 98 | 2 |
| 8 | 77 | 23 |
| 13 | 77 | 23 |
| 45 | 20 | 80 |
| 50 | 20 | 80 |
| 51 | 98 | 2 |
| 56 | 98 | 2 |

Method GRAM-ATM-1064: Column: Inertsil ODS-3 (4.6×250 mm, 5 μm; Column Temp.: 40° C.; Flow Rate: 1.0 ml/min; Injection Volume: 10 μl; Detector: UV, λ=220 nm; Run Time: 41 min; Mobile Phase A: 0.1% perchloric acid; Mobile Phase B: 100% acetonitrile; Needle wash/Diluent: 0.1% Perchloric acid:ACN=90:10 (v/v); Gradient elution:

| Time (min) | Mobile phase A (%) | Mobile phase B (%) |
|---|---|---|
| 0 | 95 | 5 |
| 25 | 64 | 36 |
| 35 | 40 | 60 |
| 36 | 95 | 5 |
| 41 | 95 | 5 |

Method 1-P-OM-WI-9091884: When analyzed DMIA-HCl, DMIA HCl-P and Pazo-2, UHPLC/MS was used, and the parameters were as follows:

| | |
|---|---|
| Parameter | Description |
| Analytical Column | Agilent Zorbax Bonus-RP Rapid Resolution 4.6 × 150 mm, 3.5 μm |
| Column Temperature | 40° C. |
| Auto Sampler Temperature | 5° C. |
| Mobile Phase A | 5 mM Ammonium Acetate in Milli-Q Water |
| Mobile Phase B | 100% Methanol |
| Flow Rate | 1.0 mL/minute |
| | Post Column: 0.5 mL/minute to MS, 0.5 mL/minute to Waste |
| Injection Volumn | 20 μL |
| Run Time | 31 minutes |

Gradient

| | | |
|---|---|---|
| Time (min) | Mobile Phase A (%) | Mobile Phase B (%) |
| 0.0 | 50 | 20 |
| 20.0 | 30 | 70 |
| 22.0 | 10 | 90 |
| 25.0 | 10 | 90 |
| 26.0 | 90 | 20 |
| 31.0 | 80 | 20 |

MS Detector Parameters

| | |
|---|---|
| Parameter | Description |
| Spray Voltage | 3000 |
| Vaporizer Temperature | 380° C. |
| Sheath Gas Pressure | 60 |
| Ion Sweep Gas Pressure | 0.0 |
| Aux Gas Pressure | 30 |
| Capillary Temperature | 380° C. |
| MS Run Time | 31 minutes |
| Scan Parameters | +p SIM Q1MS, Micro Scans 1, |
| | Center 162.000 (DMIA), Scan Width 2.000, Scan Time 1.000, |
| | Q1 Peak Width 0.70, Q3 Peak Width 0.70, S-Lens 95 |
| | Center 192.000 (DMIA-P), Scan Width 2.000, Scan Time 1.000, |
| | Q1 Peak Width 0.70, Q3 Peak Width 0.70, S-Lens 58 |
| | Center 286.000 (Pazo-2), Scan Width 2.000, Scan Time 1.000, |
| | Q1 Peak Width 0.70, Q3 Peak Width 0.70, S-Lens 80 |

When analyzed AMBF-P, UHPLC/MS was used, and the parameters were as follows:

| | |
|---|---|
| Parameter | Description |
| Analytical Column | Agilent Zorbax Bonus-RP Rapid Resolution 4.6 × 150 mm, 3.5 μm |
| Column Temperature | 40° C. |
| Auto Sampler Temperature | 5° C. |
| Mobile Phase A | 5 mM Ammonium Acetate in Milli-Q Water |
| Mobile Phase B | 100% Methanol |
| Flow Rate | 1.0 mL/minute |
| | Post Column: 0.5 mL/minute to MS, 0.5 mL/minute to Waste |
| Injection Volumn | 20 μL |
| Run Time | 31 minutes |

Gradient

| | | |
|---|---|---|
| Time (min) | Mobile Phase A (%) | Mobile Phase B (%) |
| 0.0 | 50 | 20 |
| 20.0 | 30 | 70 |
| 22.0 | 10 | 90 |
| 25.0 | 10 | 90 |
| 26.0 | 90 | 20 |
| 31.0 | 80 | 20 |

MS Detector Parameters

| | |
|---|---|
| Parameter | Description |
| Spray Voltage | 3000 |
| Vaporizer Temperature | 380° C. |
| Sheath Gas Pressure | 60 |
| Ion Sweep Gas Pressure | 0.0 |
| Aux Gas Pressure | 30 |
| Capillary Temperature | 380° C. |
| MS Run Time | 31 minutes |
| Scan Parameters | +p SIM Q1MS, Micro Scans 1, |
| | Center 162.000 (DMIA), Scan Width 2.000, Scan Time 1.000, |
| | Q1 Peak Width 0.70, Q3 Peak Width 0.70, S-Lens 95 |
| | Center 192.000 (DMIA-P), Scan Width 2.000, Scan Time 1.000, |
| | Q1 Peak Width 0.70, Q3 Peak Width 0.70, S-Lens 58 |
| | Center 286.000 (Pazo-2), Scan Width 2.000, Scan Time 1.000, |
| | Q1 Peak Width 0.70, Q3 Peak Width 0.70, S-Lens 80 |

Process GRAM-TM-0016: Endotoxin analysis with Endoscan-V software may be performed in conjunction with the Biotek EL×808 Absorbance Microplate Reader or the Endosafe Nexgen-MCS multi-cartridge system.

### RESULTS OF THE STUDY ON STABILITY

TABLE 21
Differences in characteristics of the clinical batch of the Pazopanib Hydrochloride Injection over the time
Product Code: 033-160A
Lot: K-20-097
Storage: 5° C. ± 3° C. Inverted

| Test/Method | Specification | 0M | 3M | 6M | 9M |
|---|---|---|---|---|---|
| Appearance/GRAM- | Clear, colorless | Clear, colorless | Clear, colorless | Clear, colorless | Clear, colorless |
| TM-0003 | solution | solution essentially | solution essentially | solution essentially | solution essentially |
| | essentially free | free from visible | free from visible | free from visible | free from visible |
| | from visible | particulates | particulates | particulates | particulates |
| | particulates | | | | |
| pH/GRAM-TM-0017 | 2.8-3.8 | 3.3 | 3.3 | 3.3 | 3.3 |
| Particulate | ≥10 μm: NMT | 548 particles/vial | 14 particles/ | 836 particles/ | 683 particles/ |
| Matter/USP <788> | 6000/container | | container | container | container |
| Method 1 | ≥25 μm: NMT | 7 particles/vial | 3 particles/ | 8 particles/ | 35 particles/ |
| | 600/container | | container | container | container |
| Assay (HPLC)/ | 90.0-110.0% | 101.5% | 100.3% | 100.1% | 101.4% |
| GRAM-ATM-1060 | Label Claim | | | | |
| Related Substances | DCP: NMT | <0.05% | <0.05% | <0.05% | <0.05% |
| (HPLC)/GRAM- | 0.2% | | | | |
| ATM-1063 | Pazo-1: NMT | <0.05% | <0.05% | <0.05% | <0.05% |
| | 0.2% | | | | |
| | AMBF: NMT | ND | <0.05% | ND | <0.05% |
| | 0.1% | | | | |
| | Any Other | RRT0.29:<0.05% | RRT 0.28: <0.05% | RRT 0.29: <0.05% | RRT 0.29: <0.05% |
| | Individual | RRT 0.30: <0.05% | RRT 0.30: <0.05% | RRT 0.30: <0.05% | RRT 0.30: <0.05% |
| | Impurity: | RRT 0.44: <0.05% | RRT 0.44: <0.05% | RRT 0.44: <0.05% | RRT 0.44: <0.05% |
| | NMT 0.5% | RRT 0.67: <0.05% | RRT 0.60: <0.05% | RRT 0.67: <0.05% | RRT 0.60: <0.05% |
| | | RRT 0.68: <0.05% | RRT 0.66: <0.05% | RRT 0.75: <0.05% | RRT 0.61: <0.05% |
| | | RRT 0.75: <0.05% | RRT 0.67: <0.05% | RRT 0.76: <0.05% | RRT 0.67: <0.05% |
| | | RRT 0.76: <0.05% | RRT 0.74: <0.05% | RRT 0.88: <0.05% | RRT 0.75: <0.05% |
| | | RRT 0.85: <0.05% | RRT 0.75: <0.05% | RRT 0.95: <0.05% | RRT 0.76: <0.05% |
| | | RRT 0.89: <0.05% | RRT 0.75: <0.05% | RRT 1.06: <0.05% | RRT 0.88: <0.05% |
| | | RRT 0.95: <0.05% | RRT 0.88: <0.05% | RRT 1.09: <0.05% | RRT 0.94: <0.05% |
| | | RRT 1.06: <0.05% | RRT 0.94: <0.05% | RRT 1.17: <0.05% | RRT 1.06: <0.05% |
| | | RRT 1.09: <0.05% | RRT 1.06: <0.05% | RRT 1.20 <0.05% | RRT 1.09: <0.05% |
| | | RRT 1.17: <0.05% | RRT 1.09: <0.05% | RRT 1.27: <0.05% | RRT 1.17: <0.05% |
| | | RRT 1.20: <0.05% | RRT 1.17: <0.05% | RRT 1.29: <0.05% | RRT 1.20 <0.05% |
| | | RRT 1.27: <0.05% | RRT 1.19: <0.05% | RRT 1.31: <0.05% | RRT 1.26: <0.05% |
| | | RRT 1.29: <0.05% | RRT 1.26: <0.05% | RRT 1.38: <0.05% | RRT 1.28: <0.05% |
| | | RRT 1.31: <0.05% | RRT 1.28: <0.05% | RRT 1.40: <0.05% | RRT 1.30: <0.05% |
| | | RRT 1.38: <0.05% | RRT 1.30: <0.05% | RRT 1.42: <0.05% | RRT 1.38: <0.05% |
| | | RRT 1.41: <0.05% | RRT 1.37: <0.05% | RRT 1.42: <0.05% | RRT 1.41: <0.05% |
| | | RRT 1.42: <0.05% | RRT 1.40: <0.05% | RRT 1.45: <0.05% | RRT 1.42: <0.05% |
| | | RRT 1.45: <0.05% | RRT 1.41: <0.05% | RRT 2.60: <0.05% | RRT 1.44: <0.05% |
| | | RRT 2.60: <0.05% | RRT 1.43: <0.05% | | |
| | Total | <0.05% | <0.05% | <0.05% | <0.05% |
| | Impurities | | | | |
| | (include | | | | |
| | Pazopanib-C2): | | | | |
| | NMT 1.0% | | | | |
| Related Substances | Pazopanib-C2: | <0.05% | <0.05% | <0.05% | <0.05% |
| (HPLC)/GRAM- | NMT 0.2% | | | | |
| ATM-1064 | | | | | |
| Related Substances | DMIA HCL: | ND | NA | <1.34 ppm | <1.34 ppm |
| (LC-MS)/1-P-QM- | NMT | | | | |
| WI-9091884 | 13.6 ppm | | | | |
| | AMBF-P: | ND | ND | <1.34 ppm | <1.34 ppm |
| | NMT | | | | |
| | 13.6 ppm | | | | |
| | DMIA HCL-P: | ND | ND | <1.34 ppm | <1.34 ppm |
| | NMT | | | | |
| | 13.6 ppm | | | | |
| | Pazo-2: NMT | ND | <LOQ | <1.34 ppm | <1.34 ppm |
| | 13.6 ppm | | | | |
| Sterility/USP <71> | Sterile | No evidence of | | | |
| | | microbial growth | | | |
| Bacterial Endotoxin/ | NMT 1.99 | B: <0.40 EU/mg | | | |
| USP <85> | EU/mg | M: <0.40 EU/mg | | | |
| GRAM- TM-0016 | | E: <0.40 EU/mg | | | |

TABLE 22
Differences in characteristics of the clinical batch of the Pazopanib Hydrochloride Injection over the time
Product Code: 033-160A
Lot: K-20-097
Storage: 5° C. ± 3° C. Upright

| Test/Method | Specification | 0M | 9M |
|---|---|---|---|
| Appearance/GRAM- | Clear, colorless solution | Clear, colorless | Clear, colorless solution |
| TM-0003 | essentially free from | solution free from | essentially free from |
| | visible particulates | visible particulates | visible particulates |
| pH/GRAM-TM-0017 | 2.8-3.8 | 3.3 | 3.3 |
| Particulate Matter/ | ≥10 μm: NMT | 548 particles/vial | 598 particles/container |
| USP <788> | 6000/container | | |
| Method 1 | ≥25 μm NMT | 7 particles/vial | 20 particles/container |
| | 600/container | | |
| Assay (HPLC)/GRAM- | 90.0-110.0% | 101.5% | 101.4% |
| ATM-1060 | Label Claim | | |
| Related Substances | DCP: NMT 0.2% | <0.05% | <0.05% |
| (HPLC)/GRAM- | Pazo-1: NMT 0.2% | <0.05% | <0.05% |
| ATM-1063 | AMBF: NMT 0.1% | ND | <0.05% |
| | Any Other Individual | RRT0.29 :<0.05% | RRT 0.15: <0.05% |
| | Impurity: NMT 0.5% | RRT 0.30: <0.05% | RRT 0.29: <0.05% |
| | | RRT 0.44: <0.05% | RRT 0.30: <0.05% |
| | | RRT 0.67: <0.05% | RRT 0.44: <0.05% |
| | | RRT 0.68: <0.05% | RRT 0.60: <0.05% |
| | | RRT 0.75: <0.05% | RRT 0.61: <0.05% |
| | | RRT 0.76: <0.05% | RRT 0.67: <0.05% |
| | | RRT 0.85: <0.05% | RRT 0.75: <0.05% |
| | | RRT 0.89: <0.05% | RRT 0.76: <0.05% |
| | | RRT 0.95: <0.05% | RRT 0.88: <0.05% |
| | | RRT 1.06: <0.05% | RRT 0.94: <0.05% |
| | | RRT 1.09: <0.05% | RRT 1.06: <0.05% |
| | | RRT 1.17: <0.05% | RRT 1.09: <0.05% |
| | | RRT 1.20: <0.05% | RRT 1.17: <0.05% |
| | | RRT 1.27: <0.05% | RRT 1.26: <0.05% |
| | | RRT 1.29: <0.05% | RRT 1.28: <0.05% |
| | | RRT 1.31: <0.05% | RRT 1.30: <0.05% |
| | | RRT 1.38: <0.05% | RRT 1.38: <0.05% |
| | | RRT 1.41: <0.05% | RRT 1.41 : <0.05% |
| | | RRT 1.42: <0.05% | RRT 1.42: <0.05% |
| | | RRT 1.45: <0.05% | RRT 1.44: <0.05% |
| | | RRT 2.60: <0.05% | |
| | Total Impurities | <0.05% | <0.05% |
| | (includePazopanib- | | |
| | C2): NMT 1.0% | | |
| Related Substances | Pazopanib-C2: | <0.05% | <0.05% |
| (HPLC)/GRAM- | NMT 0.2% | | |
| ATM-1064 | | | |
| Related Substances | DMIA HCL: NMT | ND | <1.34 ppm |
| (LC-MS)/1-P-QM- | 13.6 ppm | | |
| WI-9091884 | AMBF-P: NMT | ND | <1.34 ppm |
| | 13.6 ppm | | |
| | DMIA HCL-P: NMT | ND | <1.34 ppm |
| | 13.6 ppm | | |
| | Pazo-2: NMT 13.6 ppm | ND | <1.34 ppm |
| Sterility/USP <71> | Sterile | No evidence of | |
| | | microbial growth | |
| Bacterial Endotoxin/ | NMT 1.99 EU/mg | B: <0.40 EU/mg | |
| USP <85> GRAM- | | M: <0.40 EU/mg | |
| TM-0016 | | E: <0.40 EU/mg | |

TABLE 23
Differences in characteristics of the clinical batch of the
Pazopanib Hydrochloride Injection over the time
Product Code: 033-160A
Lot: K-20-097
Storage: 25° C. ± 2° C./60% RH ± 5% Inverted

| Test/Method | Specification | 0 M | 1 M | 2 M | 3 M | 6 M |
|---|---|---|---|---|---|---|
| Appearance/ | Clear, | Clear, | Clear, | Clear, | Clear, | Clear, |
| GRAM-TM-0003 | colorless | colorless | colorless | colorless | colorless | colorless |
| | solution | solution | solution | solution | solution | solution |
| | essentially | free from | free from | essentially | essentially | essentially |
| | free from | visible | visible | free from | free from | free from |
| | visible | particulates | particulates | visible | visible | visible |
| | particulates | | | particulates | particulates | particulates |
| pH/GRAM-TM-0017 | 2.8-3.8 | 3.3 | 3.3 | 3.4 | 3.4 | 3.3 |
| Particulate | ≥10 μm: NMT | 548 particles/ | 284 particles/ | 10 particles/ | 7 particles/ | 369 particles/ |
| Matter/USP <788> | 6000/container | vial | vial | vial | container | container |
| Method 1 | ≥25 μm: NMT | 7 particles/ | 9 particles/ | 2 particles/ | 2 particles/ | 9 particles/ |
| | 600/container | vial | vial | vial | container | container |
| Assay (HPLC)/ | 90.0-110.0% | 101.5% | 100.9% | 100.6% | 100.2% | 100.5% |
| GRAM-ATM-1060 | Label Claim | | | | | |
| Related Substances | DCP: NMT | <0.05% | <0.05% | <0.05% | <0.05% | <0.05% |
| (HPLC)/GRAM- | 0.2% | | | | | |
| ATM -1063 | Pazo-1: NMT | <0.05% | <0.05% | <0.05% | <0.05% | <0.05% |
| | 0.2% | | | | | |
| | AMBF: NMT | ND | ND | <0.05% | <0.05% | <0.05% |
| | 0.1% | | | | | |
| | Any Other | RRT 0.29: | RRT 0.17: | RRT 0.29: | RRT 0.26: | RRT 0.15: |
| | Individual | <0.05% | <0.05% | <0.05% | <0.05% | <0.05% |
| | Impurity: | RRT 0.30: | RRT 0.29: | RRT 0.44: | RRT 0.28: | RRT 0.17: |
| | NMT 0.5% | <0.05% | <0.05% | <0.05% | <0.05% | <0.05% |
| | | RRT 0.44: | RRT 0.30: | RRT 0.60: | RRT 0.30: | RRT 0.29: |
| | | <0.05% | <0.05% | <0.05% | <0.05% | <0.05% |
| | | RRT 0.67: | RRT 0.44: | RRT 0.61: | RRT 0.44: | RRT 0.30: |
| | | <0.05% | <0.05% | <0.05% | <0.05% | <0.05% |
| | | RRT 0.68: | RRT 0.60: | RRT 0.67: | RRT 0.60: | RRT 0.44: |
| | | <0.05% | <0.05% | <0.05% | <0.05% | <0.05% |
| | | RRT 0.75: | RRT 0.61: | RRT 0.74: | RRT 0.61: | RRT 0.61: |
| | | <0.05% | <0.05% | <0.05% | <0.05% | <0.05% |
| | | RRT 0.76: | RRT 0.68: | RRT 0.75: | RRT 0.67: | RRT 0.66: |
| | | <0.05% | <0.05% | <0.05% | <0.05% | <0.05% |
| | | RRT 0.85: | RRT 0.75: | RRT 0.76: | RRT 0.75: | RRT 0.67: |
| | | <0.05% | <0.05% | <0.05% | <0.05% | <0.05% |
| | | RRT 0.89: | RRT 0.76: | RRT 0.88: | RRT 0.75: | RRT 0.73: |
| | | <0.05% | <0.05% | <0.05% | <0.05% | <0.05% |
| | | RRT 0.95: | RRT 0.88: | RRT 0.95: | RRT 0.78: | RRT 0.74: |
| | | <0.05% | <0.05% | <0.05% | <0.05% | <0.05% |
| | | RRT 1.06: | RRT 0.95: | RRT 1.06: | RRT 0.88: | RRT 0.75: |
| | | <0.05% | <0.05% | <0.05% | <0.05% | <0.05% |
| | | RRT 1.09: | RRT 1.05: | RRT 1.09: | RRT 0.94: | RRT 0.76: |
| | | <0.05% | <0.05% | <0.05% | <0.05% | <0.05% |
| | | RRT 1.17: | RRT 1.09: | RRT 1.17: | RRT 1.06: | RRT 0.88: |
| | | <0.05% | <0.05% | <0.05% | <0.05% | <0.05% |
| | | RRT 1.20: | RRT 1.17 | RRT 1.20: | RRT 1.09: | RRT 0.95: |
| | | <0.05% | <0.05% | <0.05% | <0.05% | <0.05% |
| | | RRT 1.27: | RRT 1.20: | RRT 1.27: | RRT 1.17: | RRT 1.06: |
| | | <0.05% | <0.05% | <0.05% | <0.05% | <0.05% |
| | | RRT 1.29: | RRT 1.27: | RRT 1.29: | RRT 1.19: | RRT 1.09: |
| | | <0.05% | <0.05% | <0.05% | <0.05% | <0.05% |
| | | RRT 1.31: | RRT 1.29: | RRT 1.30: | RRT 1.26: | RRT 1.17: |
| | | <0.05% | <0.05% | <0.05% | <0.05% | <0.05% |
| | | RRT 1.38: | RRT 1.31: | RRT 1.38: | RRT 1.28: | RRT 1.20 |
| | | <0.05% | <0.05% | <0.05% | <0.05% | <0.05% |
| | | RRT 1.41: | RRT 1.39: | RRT 1.41: | RRT 1.30: | RRT 1.27: |
| | | <0.05% | <0.05% | <0.05% | <0.05% | <0.05% |
| | | RRT 1.42: | RRT 1.42: | RRT 1.42: | RRT 1.37: | RRT 1.29: |
| | | <0.05% | <0.05% | <0.05% | <0.05% | <0.05% |
| | | RRT 1.45: | RRT 1.43: | RRT 1.44: | RRT 1.40: | RRT 1.31: |
| | | <0.05% | <0.05% | <0.05% | <0.05% | <0.05% |
| | | RRT 2.60: | RRT 1.45: | RRT 2.60: | RRT 1.41: | RRT 1.38: |
| | | <0.05% | <0.05% | <0.05% | <0.05% | <0.05% |
| | | | RRT 2.61: | | RRT 1.43: | RRT 1.42: |
| | | | <0.05% | | <0.05% | <0.05% |
| | | | | | | RRT 1.42: |
| | | | | | | <0.05% |
| | | | | | | RRT 1.45: |
| | | | | | | <0.05% |
| | | | | | | RRT 2.60: |
| | | | | | | <0.05% |
| | Total | <0.05% | <0.05% | <0.05% | <0.05% | <0.05% |
| | Impurities | | | | | |
| | (include | | | | | |
| | Pazopanib-C2): | | | | | |
| | NMT 1.0% | | | | | |
| Related Substances | Pazopanib-C2: | <0.05% | <0.05% | <0.05% | <0.05% | <0.05% |
| (HPLC)/ | NMT 0.2% | | | | | |
| GRAM-ATM-1064 | | | | | | |
| Related Substances | DMIA HCL: | ND | <LOQ | NA | NA | <1.34 ppm |
| (LC-MS)/1-P-QM - | NMT 13.6 ppm | | | | | |
| WI -9091884 | AMBF-P: | ND | ND | ND | ND | <1.34 ppm |
| | NMT 13.6 ppm | | | | | |
| | DMIA HCL-P: | ND | ND | ND | ND | <1.34 ppm |
| | NMT 13.6 ppm | | | | | |
| | Pazo-2: NMT | ND | ND | ND | <LOQ | <1.34 ppm |
| | 13.6 ppm | | | | | |
| Sterility/USP <71> | Sterile | No evidence | | | | No evidence |
| | | of microbial | | | | of microbial |
| | | growth | | | | growth |
| Bacterial | NMT 1.99 | B: <0.40 | | | | <0.40 |
| Endotoxin/USP | EU/mg | EU/mg | | | | EU/mg. |
| <85> GRAM- | | M: <0.40 | | | | |
| TM-0016 | | EU/mg | | | | |
| | | E: <0.40 | | | | |
| | | EU/mg | | | | |

What is claimed is:

1. A pharmaceutical composition comprises:
pazopanib;
a cyclodextrin solubilizer; and
a solvent;
wherein the mass ratio of the pazopanib to the cyclodextrin solubilizer is 1:(13 to 100), and
based on 1 mL of the pharmaceutical composition, the concentration of the cyclodextrin solubilizer in the pharmaceutical composition is 6 to 330 mg/mL.

2. The pharmaceutical composition as defined in claim 1,
wherein the pazopanib is pazopanib hydrochloride;
or, the cyclodextrin solubilizer is a β-cyclodextrin derivative;
or, in the pharmaceutical composition, the mass ratio of the pazopanib to the cyclodextrin solubilizer is 1:(15 to 40) or 1:(80 to 100);
or, in the pharmaceutical composition, the concentration of the cyclodextrin solubilizer is 15 to 300 mg/mL,
or, in the pharmaceutical composition, the concentration of the pazopanib is 0.1 to 20 mg/mL;
or, in the pharmaceutical composition, the pH value is 2.5 to 5.5;
or, the pharmaceutical composition further comprises an additive.

3. The pharmaceutical composition as defined in claim 1,
wherein, the cyclodextrin solubilizer is a β-cyclodextrin derivative; the types of the β-cyclodextrin derivative include one or more than one of hydroxypropyl-β-cyclodextrin, sulfobutyl ether-β-cyclodextrin and methyl-β-cyclodextrin;
or, in the pharmaceutical composition, the mass ratio of the pazopanib to the cyclodextrin solubilizer is 1:(30 to 40) or 1:(90 to 100);
or, in the pharmaceutical composition, the concentration of the cyclodextrin solubilizer is 30 to 40 mg/mL or 90 to 300 mg/mL;
or, in the pharmaceutical composition, the concentration of the pazopanib is 0.5 to 2 mg/mL or 3 to 20 mg/mL;
or, when the pharmaceutical composition contains a pH regulator, the alkali in the pH regulator includes one or more than one of aqueous ammonia, sodium hydroxide, sodium carbonate and sodium bicarbonate;
or, when the pharmaceutical composition contains a pH regulator, the acid in the pH regulator includes one or more than one of phosphoric acid, hydrochloric acid, citric acid and acetic acid;
or, the pharmaceutical composition further comprises an additive, the types of the additive include one or more than one of glycerol, propylene glycol, poloxamer, sucrose, mannitol, glucose, sodium chloride and amino acids;
or, the pharmaceutical composition further comprises an additive, based on 1 mL of the pharmaceutical composition, the concentration of the additive is 0.1 to 200 mg/mL.

4. The pharmaceutical composition as defined in claim 1,
wherein the cyclodextrin solubilizer is a β-cyclodextrin derivative; the types of the β-cyclodextrin derivative include hydroxypropyl-β-cyclodextrin or sulfobutyl ether-β-cyclodextrin;
or, in the pharmaceutical composition, the mass ratio of the pazopanib to the cyclodextrin solubilizer is 1:33.26;
or, in the pharmaceutical composition, the concentration of the cyclodextrin solubilizer is 33.26 mg/mL;
or, in the pharmaceutical composition, the concentration of the pazopanib is 1 mg/mL;
or, the pharmaceutical composition further comprises an additive, based on 1 mL of the pharmaceutical composition, the concentration of the additive is 0.1 to 100 mg/mL.

5. The pharmaceutical composition as defined in claim 1,
wherein, the cyclodextrin solubilizer is a β-cyclodextrin derivative; the types of the β-cyclodextrin derivative include hydroxypropyl-β-cyclodextrin and sulfobutyl ether-β-cyclodextrin;
or, in the pharmaceutical composition, the concentration of the cyclodextrin solubilizer is 90 to 200 mg/mL or 280 to 300 mg/mL;
or, in the pharmaceutical composition, the concentration of the pazopanib is 3 to 5 mg/mL.

6. The pharmaceutical composition as defined in claim 1,
wherein the cyclodextrin solubilizer includes hydroxypropyl-β-cyclodextrin, the concentration of the hydroxypropyl-β-cyclodextrin is 15 to 300 mg/mL.

7. The pharmaceutical composition as defined in claim 1,
wherein the cyclodextrin solubilizer includes hydroxypropyl-β-cyclodextrin, the concentration of the hydroxypropyl-β-cyclodextrin is 30 to 40 mg/mL or 90 to 300 mg/mL.

8. The pharmaceutical composition as defined in claim 1,
wherein the cyclodextrin solubilizer includes hydroxypropyl-β-cyclodextrin, the concentration of the hydroxypropyl-β-cyclodextrin is 33.26 mg/mL, 90 to 200 mg/mL or 280 to 300 mg/mL.

9. The pharmaceutical composition as defined in claim 1, comprising:
pazopanib,
a cyclodextrin solubilizer, and
a solvent,
wherein the mass ratio of the pazopanib to the cyclodextrin solubilizer is 1:(15 to 100), and
based on 1 mL of the pharmaceutical composition, the concentration of the cyclodextrin solubilizer in the pharmaceutical composition is 90 to 300 mg/mL,
the types of the cyclodextrin solubilizer include hydroxypropyl-β-cyclodextrin or sulfobutyl ether-β-cyclodextrin.

10. The pharmaceutical composition as defined in claim 1, comprising:
pazopanib,
a cyclodextrin solubilizer, and
a solvent,
wherein the mass ratio of the pazopanib to the cyclodextrin solubilizer is 1:(15 to 40), and
based on 1 mL of the pharmaceutical composition, the concentration of the cyclodextrin solubilizer in the pharmaceutical composition is 90 to 300 mg/mL,
the types of the cyclodextrin solubilizer include hydroxypropyl-β-cyclodextrin or sulfobutyl ether-β-cyclodextrin.

11. The pharmaceutical composition as defined in claim 1, comprising:
pazopanib,
a cyclodextrin solubilizer, and
a solvent,
wherein the mass ratio of the pazopanib to the cyclodextrin solubilizer is 1:(90 to 100), and
based on 1 mL of the pharmaceutical composition, the concentration of the cyclodextrin solubilizer in the pharmaceutical composition is 280 to 300 mg/mL,
the types of the cyclodextrin solubilizer include hydroxypropyl-β-cyclodextrin or sulfobutyl ether-β-cyclodextrin.

12. A method for preparing the pharmaceutical composition as defined in claim 1, comprising mixing the pharmaceutical composition as defined in claim 1.

13. The method for preparing the pharmaceutical composition as defined in claim 12, wherein, the mixing is carried out by means of stirring.

14. The method for preparing the pharmaceutical composition as defined in claim 13,
wherein the mixing is performed in the order in which the pazopanib is added to the cyclodextrin solubilizer solution.

15. The method for preparing the pharmaceutical composition as defined in claim 13,
wherein the stirring time is 5 min or longer;
or, the rotation speed of the stirring is 250 rpm or higher;
or, after the mixing, sterilization is further included.

16. The method for preparing the pharmaceutical composition as defined in claim 13,
wherein the stirring time is 10 to 60 min;
or, the rotation speed of the stirring is 250 to 500 rpm;
or, after the mixing, sterilization is further included, the sterilization is a high-temperature sterilization.

17. The method for preparing the pharmaceutical composition as defined in claim 13,
wherein after the mixing, sterilization is further included, the sterilization is a high-temperature sterilization, the temperature of the high-temperature sterilization is 115° C. or above;
or, after the mixing, sterilization is further included, the sterilization is a high-temperature sterilization, the time of the high-temperature sterilization is 8 min or longer.

18. A method of treating one or more than one of acute lung injury, pulmonary fibrosis and acute respiratory distress syndrome in a subject in need thereof, comprising administering the pharmaceutical composition as defined in claim 1 to the subject.

19. An injection preparation, comprising the pharmaceutical composition as defined in claim 1.

20. A method of treating one or more than one of acute lung injury, pulmonary fibrosis and acute respiratory distress syndrome in a subject in need thereof, comprising administering the injection as defined in claim 19 to the subject.
